(19) United States
(12) Patent Application Publication
(10) Pub. No.: US 2007/0265195 A1
(43) Pub. Date: Nov. 15, 2007

(54) Multifunctional blood substitute

(76) Inventor: Daniel A. Freilich, Washington, DC (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/800,948

(22) Filed: May 8, 2007

Related U.S. Application Data
(60) Provisional application No. 60/798,992, filed on May 9, 2006.

(51) Int. Cl. A61K 38/42; A61K 38/26

(52) U.S. Cl. 514/6; 514/12

(57) ABSTRACT

A pharmaceutical formulation capable of supplying replacement blood volume and tissue oxygenation as well as other functions, such as procoagulation and pharmacological interventions, in order to enhance survivability in patients with severe blood loss. The invention also discloses a formulation and method of using the formulation in bridging severe blood loss using a multifunctional blood substitute in a prehospital setting.

### CROSS-REFERENCE TO RELATED APPLICATIONS

This application claims priority to U.S. Provisional application 60/798,992 filed May 9, 2007.

### BACKGROUND OF INVENTION

1. Field of the Invention

The invention relates to a pharmaceutical formulation capable of supplying replacement blood volume and oxygenation as well as procoagulation components to patients suffering from severe blood loss. The invention also discloses methods for the use of the pharmaceutical formulation as a bridging volume replacement for patients suffering from severe blood loss.

2. Description of the Related Art

Trauma is the leading cause of mortality among young adults and causes significant disability among survivors (Trunkey and Slater, 2000). Hemorrhage accounts for the preponderance of civilian deaths and about 28% of combat deaths. In combat, 90% of fatalities occur on the battlefield and some 50% are related to exsanguinations within minutes. Traumatic anemia due to blood loss has been directly correlated with mortality (Carson, et al., 1996). In many of these situations, blood transfusion can be life saving, pending stabilization of the patient's condition by surgery. However, the current standard of care for prehospital resuscitative fluids is crystalloids and colloids. Although blood transfusion is the preferred resuscitative choice, deployment of blood transfusion is often logistically impossible due to cost and availability of adequate blood supplies, especially in rural settings or in military combat situations. Therefore, delays in getting patients to hospital settings where blood products are available further increases mortality. Therefore, early prehospital resuscitation of hemorrhagic shock casualties is critical.

Advanced trauma life support guidelines that include rapid treatment with airway support, control of hemorrhage, aggressive crystalloid fluid replacement and rapid evaluation of traumatic victims have increased survival. Colloid and hypertoxin saline fluids may be an improvement over crystalloid fluid infusion. However, attempting to increase oxygen delivery by increasing only O₂ saturation (supplemental O₂) is clearly insufficient. Crystalloid fluid resuscitation increases systemic flow, however, considerable deficits in microcirculatory end organ perfusion and tissue oxygenation persist, leading to common complications including multi-organ failure. Therefore, maximizing oxygen delivery by replenishment of oxygen content with hemoglobin is paramount (Ivatury and Sugerman, 2000; Ba, et al., 2000). Thus, safe and efficacious oxygen carrying resuscitative fluid is urgently needed to decrease potentially avoidable fatalities and enhancement of survivability following trauma.

New methods to address hypotensive or low volume resuscitation may maximize the benefits of fluid replacement and minimize its adverse consequences. One approach is the utilization of hemoglobin based oxygen carriers (HBOCs). HBOCs are chemically modified hemoglobin solutions containing polymerized, conjugated or liposome or nanoparticle encapsulated hemoglobin. Earlier iterations of hemoglobin substitutes has been somewhat disappointing due to nitric oxide scavenging and consequent vasoactivity (Doherty, et al., 1998; Liao, et al., 1999), free radical generation and exacerbation of reperfusion injury, methemoglobin production and immunological effects including immunosuppression and potentiation of endotoxin-related pathogenicity (Chang, et al., 1998; Chang, et al., 2000; Su, et al., 1997). Thus far, demonstration of clinical equivalence with blood transfusion has been elusive (Sloan, et al., 1999).

Complications due to shock and resuscitation, including free radical generation and immune activation, cause reperfusion injury, multi-organ failure and delayed mortality. In ischemic tissues, hypoxantihine is produced and xanthine oxidase is activated. Reperfusion of ischemic tissue with oxygen results in the conversion of hypoxanthine into superoxide by xanthine oxidase. Superoxide results in oxygen radical formation, leading to reperfusion tissue injury. Amelioration of the effects of radical formation, and therefore tissue damage, is the use of antioxidants. In red blood cells, superoxide dismutase (SOD) and catalase convert superoxide to hydrogen peroxide and subsequently into water and oxygen. Although the half-life of SOD and catalase is brief (i.e. seconds), therefore making exogenous administration impractical.

However, heme oxygenation, free radical generation and reperfusion injury characteristic of earlier products have been decreased by the development of a method to cross-link hemoglobin with SOD and catalase using gluteraldhyde polymerization (polyHb-SOD-CAT) (Chang, et al., 2000; D'Agnillo and Chang, 1997; D'Agnillo and Chang, 1998). Alternative products include o-adenosine intra- and inter-molecular hemoglobin cross-linking and combination with reduced glutathione (Simoni, et al 1998); and by molecular modification of the heme site to yield reduced NO reactivity by heme site-directed mutagenesis (Doherty, et al., 1998); polynitroxylated hemoglobin (Buehler, et al., 2004). Vasoactivity can also be decreased by encapsulation of modified hemoglobin in liposomes and polylactic polyglycolide biodegradable nonparticle polymers (Chang, et al., 2000).

Important in blood substitute efficacy are the inclusion of multiple functions to the blood product. Blood products would be indicated for a range of patients suffering from severe trauma with concomitant hemorrhage. Therefore, artificial blood products, with not only tissue oxygenation capacity but ones that also fulfills a procoagulation role are critically needed.

Recombinant factor VIIa (rfVIIa) has been approved by the Food and Drug Administration for hemostatic indications in hemophiliac patients with inhibitors of factors VIII and IX. For that indication, is has demonstrated utility for decreasing prothrombin time, partial thromboplastin time, and bleeding, and appears safe (Hedner, 2001; Jurlander, et al., 2001). Recombinant factor VIIa has also been demonstrated to have potential utility in increasing hemostasis in uncontrolled hemorrhage.

Although the mechanism of action of rfVIIa is unknown, the presence of tissue factor or factors Xa and IXa on the endothelium is required for the potency of rfVIIa and explains the thrombotic activity limited to sites of injury and not generalized. It appears that thrombin on activated platelets may explain high-level binding of rfVIIa to platelet surfaces (Kenet, et al., 1999). Thrombotic activity appears to be limited to sites of injury and not generalized. Furthermore, in a severe swine hypothermic coagulopathic uncontrolled traumatic hemorrhage model, rfVIIa showed significant efficacy and no evidence of macro- or microthrombi in the lung (Martinowitz, et al., 2001 (I and II)).

It has been observed that infusible platelet membranes (IPM), produced from outdated or separated/centrifuged human platelets retain procoagulant activity (Chao, et al., 1996). These platelets, however, lack ABO and HLA-related immunogenicity. Nevertheless, functional hemostatic activity was demonstrated in animal models, despite absence of aggregative properties of platelets due to loss of cellular function (Galan, et al., 2000; Chao, et al. 1996). IPM procoagulant activity has been shown in the Baumgartner flow system, the thrombin generation assay, and in vivo in thrombocytopenic rabbit bleeding time assays (Chao, et al., 1996). Lack of thrombotic activity land efficacy in thrombocytopenic patients, has been demonstrated in Phase I and II clinical trials. Additionally, IPMs can be heat- or chemically-sterilized and freeze-dried. IPMs have been stored for four years at 4° C. and still retained in vitro properties (Aster, et al., 1997; Chao, et al. 1996; Escolar, et al., 1994; Enright, 1997; Scigliano, 1997). Therefore, it has been suggested that IPMs are a potentially useful procoagulant in situations where whole blood is not readily available, such as in battlefield situations.

### SUMMARY OF INVENTION

Artificial blood products are critically needed to augment or supplant natural blood products in the treatment of trauma patients especially those suffering from hemorrhagic shock. The blood product must be capable not only capable of reestablishing blood volume but also provide adequate oxygenation to tissue and to possess procoagulation factors.

Therefore, an object of this invention is a method of providing a bridging replacement fluid to trauma patients by the administration of a multi-functional blood substitute capable of providing tissue oxygenation as well as promoting coagulation.

Another object of the invention is a multifunctional blood substitute (MBS) comprising a modified hemoglobin and one or more of procoagulation factors including recombinant procoagulation factors and infusible platelet membranes, anti-inflammatory agents and nitric oxide donors.

### BRIEF DESCRIPTION OF DRAWINGS

FIG. 1. Measurement of hemostasis parameters of MBS, rfVIIa and HBOC.

FIG. 2. Survival rates in thromobcytopenic rabbits following administration of lactate ringers (LR), infusible platelet membranes (IPM), HBOC, rfVIIa and MBS.

FIG. 3. Systemic and pulmonary pressures and vascular resistance in swine administered varying levels of tetrameric hemoglobin HBOC.

FIG. 4. Blood loss in swine following liver injury.

FIG. 5. Tissue oxygenation provided by HBOC.

FIG. 6. Blood loss in swine administered HBOC plus rfVIIa

FIG. 7. Hematology of swine administered HBOC plus rfVIIa

FIG. 8. Survival of swine following liver injury administered HBOC plus rfVIIa.

### DESCRIPTION OF THE PREFERRED EMBODIMENTS

The current invention provides an improved formulation and method of its use for the treatment of patients suffering from hemorrhagic shock. The inventive formulation is also useful in providing a whole blood-like “bridging” volume replacement fluid with oxygen transporting properties as well as hemostatic, immunomodulating and antiapoptotic and antioxidant properties. Therefore, the inventive method, using the inventive formulation, permits stabilization of patients suffering from severe blood loss until the patient can be transported to locations where life-saving transfusions and surgical procedures can be undertaken.

Improvements in the side effects of modified hemoglobin center on enhancing the hemoglobin substitute's therapeutic/toxic ratio. A comprehensive approach to reducing HBOC vasoactivity is important in order to lower low molecular weight (MW) components (dimeric and tetrameric hemoglobin (32 and 64 kdal), increasing high MW components (greater than 256-512 kdal), latering P50, increasing the hemoglobin concentration, and adding pharmacological nitric oxide donors (e.g., nitroglyerine, L-arginine) (Fischer, et al., 1999) and/or drag reducing polymers (Kameneva, et al., 2004).

Alternative methods to reduce vasoactivity include molecular modification of the heme site to yield reducing NO reactivity by heme site-directed mutagenesis (Doherty, et al., 1998); polynitroxylation of hemoglobin (Buehler, et al., 2004); encapsulation of modified hemoglobin in liposomes and polylactic polyglycolide biodegradable nonparticle polymers (Chang, et al., 2000); and zero-linked mega high MW hemoglobin (approx. 25 Mdal)(Bucci, et al., 2007). Arachidonic acid agaonists (e.g., non-steroidal anti-inflammatory drugs (NSAIDS) may also be utilized to counteract pulmonary vasoactivity.

Additional methods to diminish HBOC-related immune activation (and apoptosis) include amelioration of the excipient (solution). Some the approaches will include substituion of racemic Lacted Ringers's (dl-LR) solution with l-LR or ketone-LR (Alam, et al., 2004; Doustova, et al., 2003).

Hemostasis, the arrest of bleeding from an injured blood vessel, involves the combined activity of a number of blood components including vascular, platelet and plasma factors. Vascular factors reduce blood flow from trauma by local vasoconstriction, in response to injury, and compression of injured vessels by blood extravasated into surrounding tissues.

Recombinant factor VIa has also been demonstrated to have potential utility in increasing hemostasis in uncontrolled hemorrhage. Therefore, inclusion of rfVIIa as a component of a multifunction blood substitute serves to improve hemostasis in a hemorrahagic patient and is, therefore, highly warranted. Furthermore, given the ability of IPM to be heat- or chemically-sterilized and stored freeze-dried yet still retain their procoagulant properties, makes their addition as a component of multifunctional blood substitutes practical.

In addition to procoagulation factors, an inventive aspect is an MBS formulation that contains one or more pharmacologic interventions. These interventions are selected based on their ability to counteract potential adverse effects of hemorrhagic shock, reperfusion injury and potential HBOC toxicities and include nitric oxide donors (i.e. anti-vasoactive), such as nitroglycerine, L-arginine; free radical scavengers and immunomodulators, such as mannitol, superoxide dismutase and catalase, 17-hydroxyimmunosteroids, anti-CD18, pentoxifylline, 2-mercaptopropionyl glycine, edaravone (MCI-186), melatonin, mercaptoethylguanidine, intracellular adhesion molecules (ICAM) and vascular cell adhesion molecule (VCAM).

Although the example given utilized gluteraldehyde crosslinked bovine hemoglobin other HBOC formulations are contemplated to be able to be substituted in the MBS. A preferred embodiment of the invention is the utilization of gluteradldehyde cross-linked bovine hemoglobin-superoxide dismutase-catalase (polyHb-SOD-catalase). PolyHb-SOD-catalase has been shown to decrease free radicals and Fe release in vitro and in rat reperfusion models. Therefore, PolyHb-SOD-catalase will likely be effective at decreasing toxicity of HBOC due to oxidant damage and multi-organ failure. PolyHb-SOD-catalase has been shown to decrease 3,4 dihyroxthenzoate (3,4 DHBA) levels in intestinal effluent (Chang, et al., 2000; D'Agnillo and Chang, 1997; D'Agnillo and Chang, 1998). Therefore, maximum benefit of HBOC can be afforded by inclusion of an HBOC formulation that minimizes tissue damage and toxicity along with other pharmacologics that further mitigate or abrogate potential HBOC effects and procoagulation factors.

### EXAMPLE 1

### Example of Embodiment of Multifunctional Blood Substitute in Rabbit Model

Clinical utility of oxygenating an artificial blood substitute is significantly enhanced by the addition of procoagulation factors. An example of the contemplated invention is a MBS, comprising a HBOC, such as bovine polymerized hemoglobin and the procoagulation factor rfVIIa with or without infusible platelet membranes (IPM). In this embodiment, the HBOC is either inter- and/or intramolecularly crosslinked.

Alternatively, the hemoglobin can be conjugated to another molecule such as glutaraldhyde or polyethylene glycol (U.S. Pat. No. 5,905,141 to Rausch, et al; U.S. Pat. No. 618,919 to Rausch, et al; U.S. Pat. No. 5,084,558 to Rausch, et al; U.S. Pat. No. 5,296,465 to Rausch, et al; U.S. Pat. No. 5,840,852 to Rausch, et al; U.S. Pat. No. 5,753,616 to Rausch, et al; U.S. Pat. No. 5,895,810 to light, et al; U.S. Pat. No. 5,691,452 to Gawryl, et al; U.S. Pat. No. 5,691,453 to Wertz, et al; and U.S. Pat. No. 5,808,011 to Gawryl, et al).

In a preferred embodiment, HBOC consists of glutaraldehyde-polymerized, ultra purified bovine hemoglobin (Biopure Corp., Cambridge, Mass.). The crosslinked product has a mw greater than 500 kd. Greater than 95% of the HBOC is contributed by multimeric HB as octomers or greater (i.e. less than 5% tetrameric hemoglobin). The HBOC had a P₅₀ of approximately 37 torr (Hill coefficient of approximately 1.4). The endotoxin level of the HBOC solution is 0.5 endotoxin units per mil or less. In the preferred embodiment, rfVIIa is administered at a dose of 45 ug/kg to 360 μg/kg and IPM was administered at 6 mg/kg. A preferred embodiment may also include MBS modifications including multiple approaches to reduction of vasoactivity (e.g., lowered dimeric/tetrameric HB content, high-MW HB content, altered P₅₀, higher HB concentration and addition of NO donors or drag reducing polymers), improved excipient (e.g., l-LR replacement of dl-LR), and a NSAID to counteract pulmonary vasoactivity.

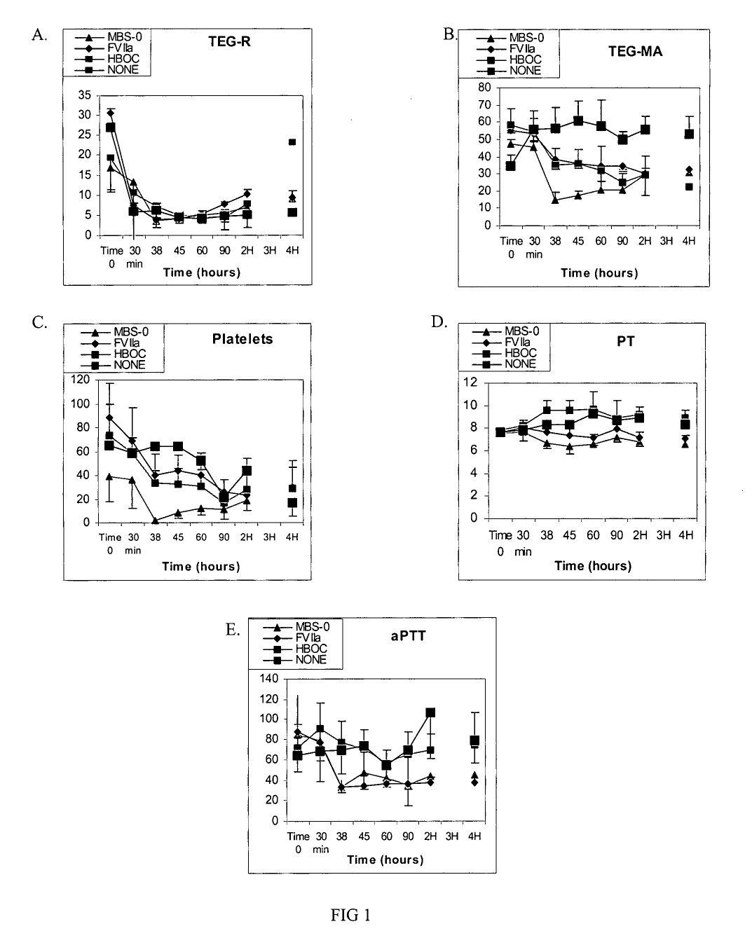

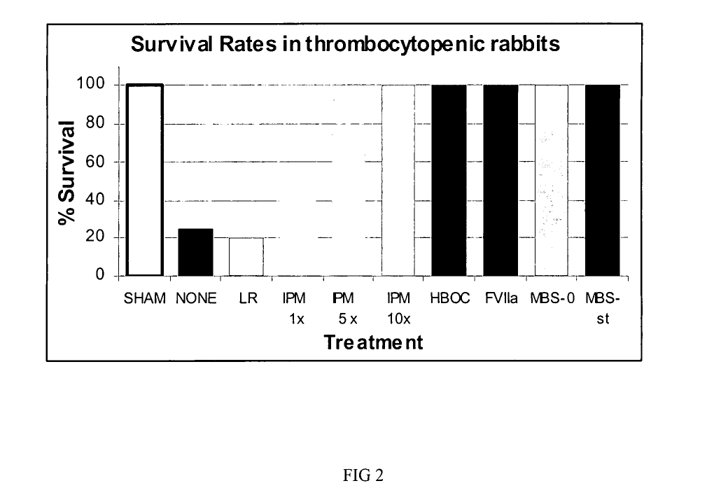

As shown in FIG. 1, in rabbits, inclusion of procoagulation factors with HBOC leads to markedly improved hemostatic properties as indicated in thromboelastography “r” time (FIG. 1(A)) and maximum amplitude (TEG-MA) (FIG. 1(B)). Referring to FIG. 1 (D) and (E), to further illustrate the invention, thrombin and prothrombin time was also significantly reduced in MBS verses HBOC. In FIG. 1, MBS-0 refers to freshly produced, verses previously stored, MBS. Additionally, in studies of controlled hemorrhage in rabbits, the survival rates of thrombocytopenic animals were significantly improved over LR by the administration of the procoagulaton factor IPM or rfVIIa or with administration of HBOC or MBS-0 or MBS-st (previously stored MBS) (FIG. 2). Furthermore, there was a significant benefit regarding survivability in utilizing IPM. The stability of MBS is illustrated in FIG. 2 since the survivability of the animals using MBS prepared fresh (MBS-0) was equivalent to that observed using MBS that had been stored for up to 6 months at 4° C. (MBS-st).

Although the example given utilized gluteraldehyde-crosslinked bovine hemoglobin other HBOC formulations are contemplated to be able to be substituted in the MBS. A preferred embodiment of the invention is the utilization of gluteraldehyde cross-linked bovine hemoglobin-superoxide dismutase-catalase (polyHb-SOD-catalase). PolyHb-SOD-catalase has been shown to decrease free radicals and Fe release in vitro and in rat reperfusion models.

### EXAMPLE 2

### Example of Embodiment of Multifunctional Blood Substitute in Swine Model

Studies were designed to mimic circumstances surrounding hemorrhagic shock using controlled hemorrhage and uncontrolled hemorrhage models. In these models, severity-escalation and evacuation delay-escalation with accompanying tissue and/or organ injuries was conducted. Like in Example 1, MBS comprised a HBOC, such as bovine polymerized hemoglobin and the procoagulation factor rfVIIa with infusible platelet membranes (IPM).

In controlled hemorrhage model studies, pigs were randomly assigned into one of three resuscitation fluid subgroups, HBOC, Hetastarch (HEX), and no therapy in one of three blood volume/time delay cohorts. The reader is referred to Table 1 for study design. There were a total of 8 pigs in each subgroup for a total of 24 animals in the mild and moderate and delay cohorts. The HBOC, Hetastarch and control (no therapy) resuscitation fluids were tested at two estimated blood withdrawal conditions (40% and 55%) and two evacuation delays (4 and 24 hours). The studies simulate the clinical scenario of an injury for which hemostasis is achieved after extensive life-threatening hemorrhage has already occurred (e.g. clamping the femoral artery after a leg amputation). In these studies, swine underwent a soft issue injury, volume controlled hemorrhage, fluid resuscitation and four hours of hemodynamic monitoring while under anesthesia.

In anesthetized, invasively monitored swine, vital signs, O₂ saturation, transcutaneous tissue oxygenation, end tidal carbon dioxide, body temperature, pulmonary catheter (Swanz-Ganz) parameters, cardiac output and urine output was measured to establish baseline values. A soft tissue crush injury (rectus abdominus muscle) and volume-controlled hemorrhage was performed. Five minutes after hemorrhage, fluid resuscitation was commenced using one of four resuscitation therapies, as described above. After the monitoring phase was complete, the animals in the mild delay (4 hours) cohorts were administered treatment that would occur with definitive medical care (i.e. normal saline or shed whole blood was infused based on specific experimental transfusion trigger parameters, administered analgesia and antibiotic, and recovered from anesthesia. Animals in the moderate delay (24 hours) were administered analgesia, antibiotic and recovered from anesthesia. Animals in these cohorts were administered treatment that would occur with definitive medical care (i.e. normal saline or shed whole blood infused based on specific experimental transfusion trigger parameters) at either approximately 24 hours (moderate delay) after the initial hemorrhage and injury.

All animals were monitored post-operatively (including additional analgesia and antibiotics) and provided additional fluid therapy per the experimental design until the 72-hour end-of-study time point when they were sedated for final blood collection, euthanasia and tissue collection. Blood samples were collected on a predetermined schedule and analyzed throughout the course of each experiment.

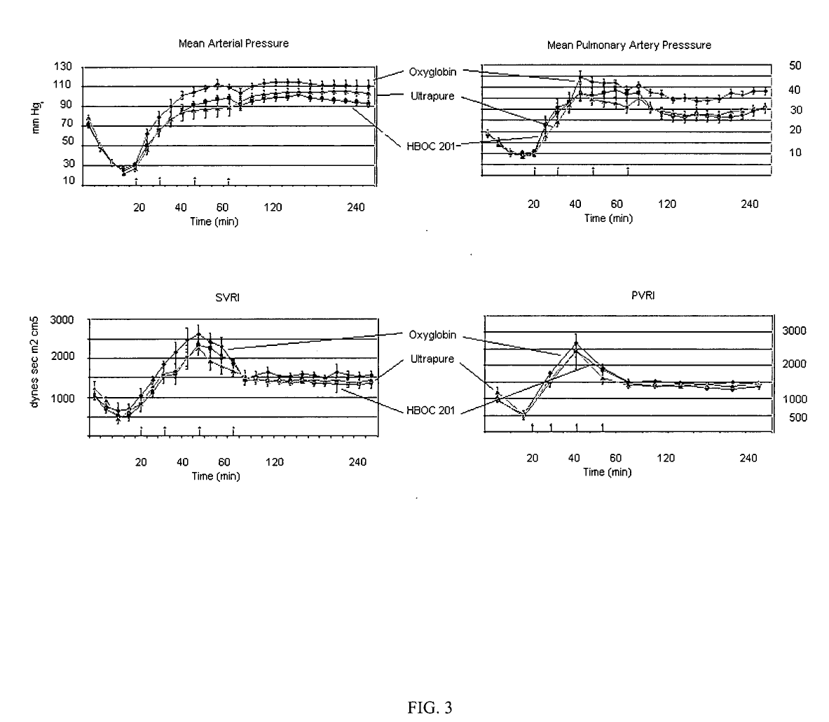

Referring to FIG. 3, a low tetrameric hemoglobin (less than 1% tetrameric Hb) solution (Ultrapure) was tested in a similar 55% controlled hemorrhage model in comparision to HBOC-201 (1-3% tetrameric hemoglobin) and Oxyglobin (˜30% tetrameric hemoglobin. Pigs were resuscitated with four infusions of 10 ml/kg of the test solution and monitored for 4 hours. Blood or saline was provided every 30 minutes from 60 minutes to 4 hours. Mean arterial blood pressure (MAP) and mean pulmonary arterial pressure were not different between HBOC-201 and Ultrapure. However, pressures were lower in these groups in comparison to Oxyglobin. Similarly, systemic vascular resistance index (SVRI) was higher in pigs resuscitated with Oxyglobin but not different between those given HBOC-201 or Ultrapure. Pulmonary vascualar resistance (PVRI) was not different between the groups. Swine were euthanized at 4 hours and long term survival was not evaluated.

TABLE 1
Summary of volume-controlled hemorrhagic shock experiments

| Experiment | Estimated | Resuscitation | Mild delay | Moderate delay |
|---|---|---|---|---|
| Arm¹ | Blood Volume | Therapy | (4 hours) | (24 hours) |
| A | 40% | HBOC | 8 | — |
| | | HEX | 8 | — |
| | | None | 8 | — |
| B | 55% | HBOC | 8 | 8 |
| | | Hetastarch | 8 | 8 |
| | | None | 8 | 8 |

¹Total pigs = 72

An additional experiment focused on establishing the therapeutic potency and breadth of BPH and MBS for the resuscitation of uncontrolled hemorrhagic shock casualties in combat and space-travel relevant scenarios (Katz et al, 2002). The experimental phase of this protocol consisted of four parts: liver crush injury, uncontrolled hemorrhage, resuscitation, and definitive stabilization.

In the uncontrolled hemorrhagic shock model studies, 34 pigs were used. The reader is referred to Table 2 for a summary of study design. A standardized liver injury was created by placing a ring clamp over the left lower lobe, ˜50% in width and ˜0.75-2.0″ from the apex, adjusting for relative size of the liver and weight of the pig. The clamp was closed and an 11 blade was used to lacerate the lobe from the top of the clamp through the remaining width. The liver injury denoted the start of the pre-hospital phase (Time 0). After 1 minute, the clamp was removed and the remaining tissue excised, resulting in 25% lobectomy, consistent with a grade III liver injury. Bleeding was spontaneous, removed via intraperitoneal suction, and quantified by weight. The pigs were randomly divided into four resuscitation subgroups, 26 animals for each of three resuscitation fluids (HBOC, Hetastarch, and MBS) and 8 animals for the subgroup that received no resuscitation fluids.

TABLE 2
Summary of uncontrolled hemorrhagic shock experiments.

| Experiment Arm | Resuscitation Therapy | Moderate delay |
|---|---|---|
| C | HBOC | 8 |
| | HEX | 8 |
| | MBS | 10 |
| | None | 8 |

¹Total pigs = 34

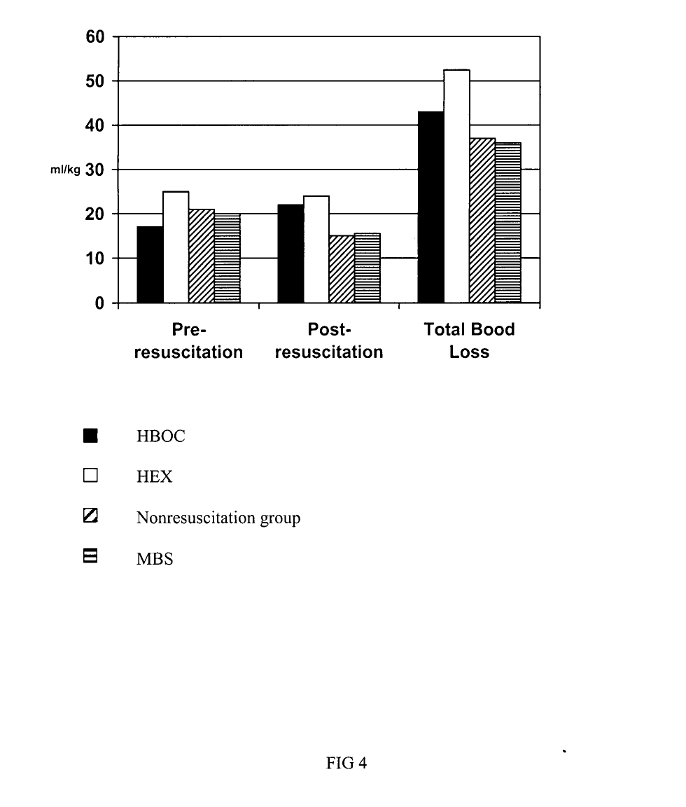

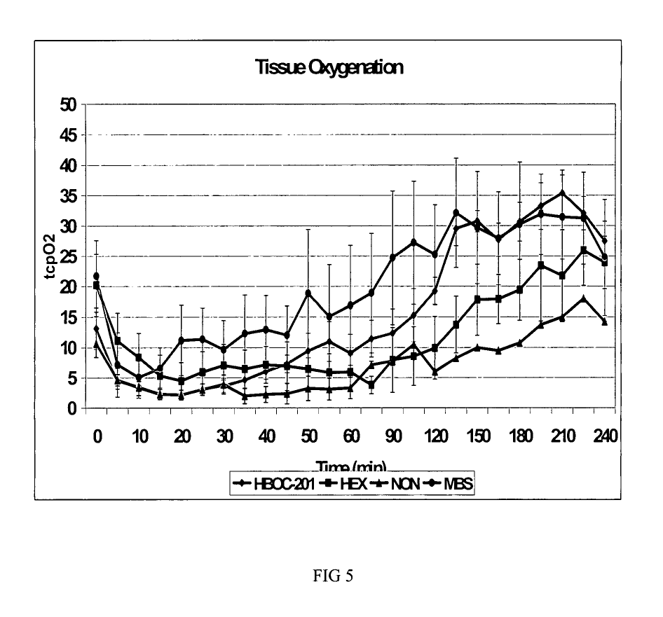

Referring to FIG. 4, blood loss in swine following liver injury was markedly reduced over HBOC alone. Unlike other asanguinous solutions, however, such as Hextend, MBS, like HBOC, has the ability to provide oxygenation to tissues. FIG. 5 illustrates the tissue oxygenating properties of MBS, in comparison to HBOC and 6% hetastarch in balanced salt solution (HEX). In FIG. 5, “NON” refers to a no resuscitation group. As illustrated by FIG. 5, HBOC and MBS provide an improved tissue oxygenation over time compared to HEX and NON.

### EXAMPLE 3

### MBS Containing HBOC Plus Recombinant Factor VIIa

Studies using HBOC plus rfVIIa (HBOC/F7), but without IPM, were conducted in swine with liver injury in order to evaluate the efficacy of rfVIIa (F7), used in combination with a hemoglobin based oxygen carrier (HBOC-201, Biopure Corp, MA) for resuscitation of hemorrhagic shock.

HBOC-201 is purified, filtered, stroma free and heat-treated bovine Hb that is polymerized by gluteradehyde-crosslinking to form polymers ranging from 130-500 kd MW. The HBOC is prepared in a buffer similar to lactated Ringer's solution and contains approximately 13 g Hb/dL.

The preparation of the F7 given to the treated experimental group was 9 ug F7/ml HBOC, 18 ug F7/ml HBOC and 36 ug F7/ml HBOC for the 90 ug/kg (1×), 180 ug/kg (2×), and 360 ug/kg (4×). Two bags of HBOC-201 were prepared (500 ml) with the appropriate amount of 2.4 mg F7 vials (i.e. 2, 4, or 8 vials were reconstituted for 1×, 2× and 4× respectively, left over was kept at 4° C.) manually injected into the bags under sterile conditions. If additional volumes of fluid were required, the exact volume was prepared in a sterile syringe to avoid wasting F7 or HBOC.

A standardized liver injury was created as described previously for uncontrolled studies in Example 2. Pigs were then randomly allocated to 1 of 4 treatment groups: Hemoglobin based oxygen carrier (HBOC-201) was compared with HBOC-201 plus increasing doses of rfVIIa (HBOC/F7 90 ug/kg, HBOC/F7 180 ug/kg, HBOC/F7 360 ug/kg). Fifteen minutes into uncontrolled hemorrhage, resuscitated pigs were administered 10 ml/kg of test fluid. At 30 minutes, an additional infusion of 5 ml/kg was administered. Additional infusions were provided at 60, 120, and 180 minutes post-injury if hypotension (MAP<60 mm Hg) or tachycardia (HR>baseline value [Time 0]) were observed. Fluids were infused over 10 minutes at room temperature.

Hospital arrival was simulated at 4 hours. Animals were administered 10 ml/kg allogeneic packed red blood cells (PRBC) for anemia (Hb<7 g/dL) and/or 10-20 ml/kg normal saline (NS) for hypotension. 13 mg/kg cephazolin (Ancef [antibiotic]), and 0.01 mg/kg buprenorphine (analgesic), were administered. The PAC was removed and the jugular vein introducer was secured for postoperative blood sampling and fluid administration. The arterial and bladder catheters removed and areas repaired as necessary. Surgical incisions were closed and surgical dressings applied. Animals were extubated and recovered from anesthesia. Vital signs and general status were assessed 24, 48, and 72 hours post-injury. Pigs received 10 ml/kg NS or PRBCs as needed for anemia or hypotension as well as antibiotics and analgesia.

Pigs were euthanized 72 hours post-injury for necropsy and histological analysis. Euthanasia was performed in accordance with the current American Veterinary Medical Association (AVMA) guidelines. Final blood samples were drawn through the indwelling central venous catheter and final non-invasive hemodynamic measurements were taken. Necropsy was performed on 72 hour “long-term” survivors and early deaths. Complete gross evaluations were performed and severity semi-quantitatively scored. LM histopathologic lesions were identified, recorded, and semi-quantitatively scored. Lesion scores were based on percentage of tissue involvement and severity of cellular changes. Additionally, standardized lung sections were also collected and fixed and examined by LM, and thin (90 nm) sections stained with lead citrate and uranyl acetate and examined with a LEO 912 AB electron microscope (LEO Electron Microscopy, Thornwood, N.Y.). Thrombosis and hemostasis was assessed by standard tests on blood samples collected at 0, 30, 60, 180 and 240 minutes, and 24, 48, and 72 hours. The results of the study demonstrated that all treatments stabilized hemodynamics.

Mean arterial pressure (MAP) differed significantly between treatment groups in the pre-hospital phase (p=0.0424). In all groups, MAP decreased during hemorrhage and was restored to near baseline values following resuscitation. MAP was restored to baseline at 120 minutes in HBOC group, at 45 minutes in the HBOC/F7 90 ug/kg group, and at 105 minutes in the HBOC/F7 180 ug/kg, whereas it did not return to baseline until 195 minutes in the HBOC/F7 360 ug/kg pigs. MPAP was similar between groups throughout the pre-hospital period p=0.4956). Heart rate (HR) was significantly different between groups over time (p=0.0027). HR increased in all groups in response to hemorrhage but fell in all groups except the HBOC/F7 180 ug/kg. In this group, HR continued to increase until 120 minutes at which point the tachycardia began to resolve. HR in the HBOC/F7 180 ug/kg remained elevated in comparison to the other groups until the end of the pre-hospital phase. Cardiac index (CI) was lowest in HBOC pigs throughout the pre-hospital period and never returned to baseline. CI was similar between other treatment groups and was restored to baseline at 135 minutes in the HBOC/F7 90 ug/kg group, at 195 minutes in the HBOC/F7 180 ug/kg group, and approached baseline in the HBOC/F7 360 ug/kg by the end of the pre-hospital period (p<0.0001).

Lactate increased in all groups in response to hemorrhage and remained above baseline in all groups with the exception of the HBOC/F7 90 ug/kg group. A substantial increase in lactate was observed in the HBOC/F7 360 ug/kg group at 150 and 180 minutes. Arterial O₂ (SaO₂) and mixed venous O₂ (SvO₂) were similar between groups during the pre-hospital period (p=NS). Base excess was lowest in the HBOC/F7 180 ug/kg group from the start of resuscitation to 120 minutes and highest in the HBOC/F7 90 ug/kg group from 60 minutes. Due to technical problems, no BE data was collected for the HBOC/F7 360 ug/kg group. Although O₂ delivery (DO₂) was not significantly different between groups, it was consistently higher in the HBOC/F7 360 ug/kg group during the prehospital phase. No differences in O₂ consumption (VO₂) or O₂ extraction ratio (O₂ER) were observed. Transcutaneous oxygen saturation (tcpO₂) was significantly different between groups over time (p<0.0001). Values were highest in the HBOC/F7 180 ug/kg group from 60 minutes and lowest in the HBOC group (45 to 75 minutes) and HBOC/F7 360 ug/kg group (105 to 210 minutes).

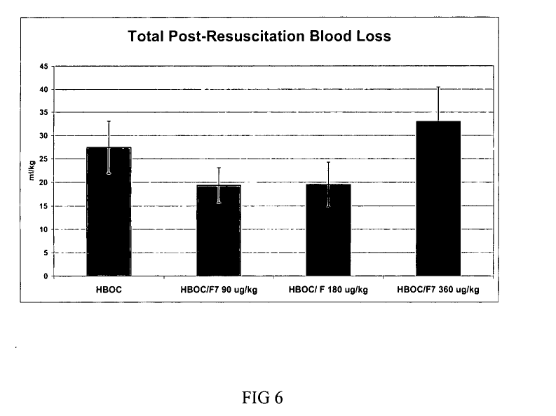

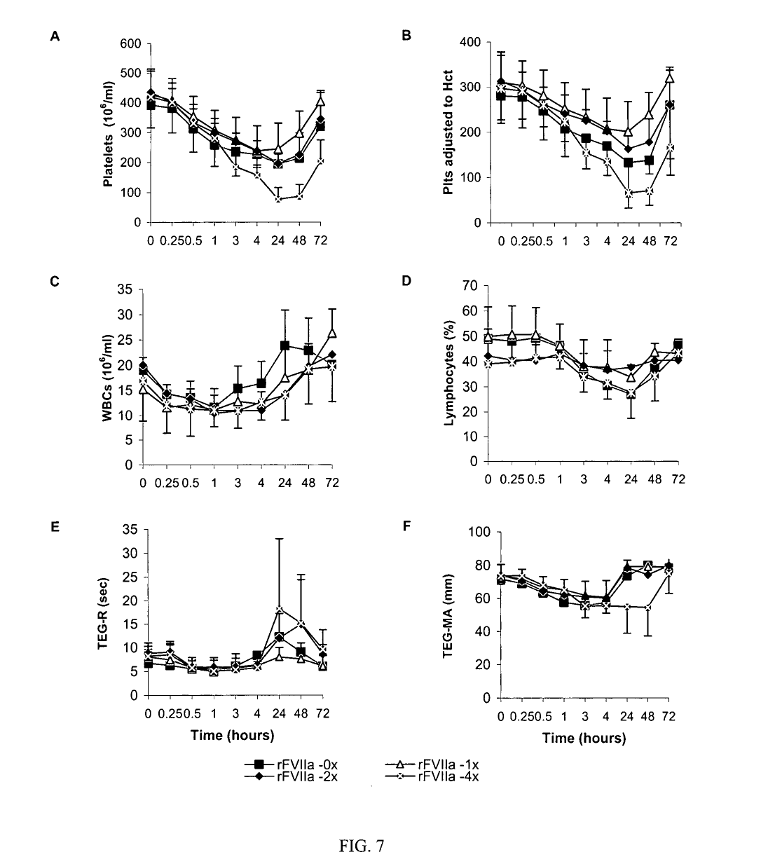

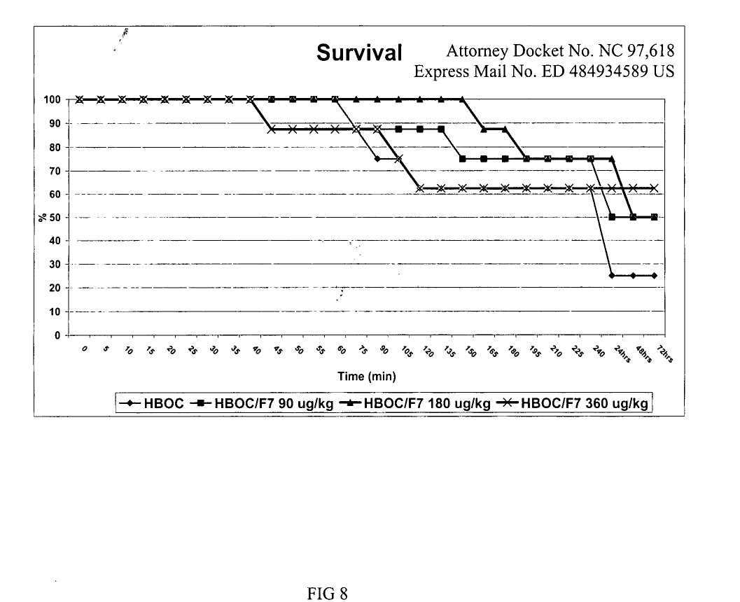

There were no significant differences in blood loss between treatment groups (refer to FIG. 6). Nor was there a difference in bleeding time between groups. F7 did affect some in vitro assays used to monitor hematology (e.g. CBC) and hemostasis (e.g. TEG, PGA, coagulation). The reader is referred to FIG. 7. However, an improved survival to simulated hospital arrival (240 minutes) was observed in the 90 μg/kg and 180 μg/kg groups. However, survival to 72 hours was not different between groups. The reader is referred to FIG. 8. There was a trend to higher survival time in the HBOC/F7 pigs (p=0.09).

### REFERENCES

1. Alam, H. B., K. Standton, E. Koustova, D. Burris, N. Rich, P. Rhee. 2004. Effect of different resuscitation strategies on neutrophil activation in a swine model of hemorrhagic shock. Resuscitation. 60:91-9.
2. Aster, R. H. 1997. Cyplex platelet alternative (infusible platelet membranes)-preclinical characterization. Current advances in platelet substitutes, Preceding the 39^{th} Annual Meeting of the American Society of Hematology, San Diego, Calif., Dec. 5, 1997.
3. Ba, Z. F., P. Wang, D. J. Koo, W. G. Cioffi, K. I. Bland, I. H. Chaudry. 2000. Alterations in tissue oxygen consumption and extraction after trauma and hemorrhagic shock. Crit Care Med. 28:3102-3103.
4. Bucci, E., H. Kwansa, R. C. Koehler, B. Matheson. 2007. Development of zero-link polymers of hemoglobin, which do not extravasate and do not induce pressure increases upon infusion. Artif Cells blood Substit Immobil Biotechnol. 35:11-18.
5. Buehler, P. W., C. R. Haney, A. Gulati, L. Ma, C. J. Hsia. 2004. Polynitroxyl hemoglobin: a pharmacokinetic study of covalently bound nitroxides to hemoglobin platforms. Free Radic Biol Med. 37:124-135.
6. Carson, J. L., A. Duff, R. M. Poses, J. A. Berlin, R. K. Spence, R. Trout, H. Noveck, B. L. Strom. 1996. Effect of anaemia and cardiovascular disease on surgical mortality and morbidity. Lancet 348:1055-1060.
7. Chao, F. C., B. K. Kim, A. M. Houranieh, F. H. Liang, M. W. Konrad, S. N. Swisher, J. L. Tullis. 1996. Infusible platelet membrane microvesicles: a potential transfusion substitute for platelets. Transfusion. 36:536-542.
8. Chang, T. M. S. (ed.). 1998. Blood substitutes: principles, methods, products and clinical trials, Vol. II. Karger, Basel, Switzerland.
9. Chang, T. M., F. D'Agnillo, W. P. Yu, S. Razack. 2000. Two future generations of blood substitutes based on polyhemoglobin-SOD-catalase and nanoencapsulation. Ad. Drug Deliv Rev. 40:213-218.
10. D'Agnillo, F. and T. M. S. Chang. 1997. Reduction of hydroxyl radical generatin in a rat hindlimb model of ischemia-reprefusion injury using crosslinked hemoglobin-superoxide dismutase-catalase. Artif Cells Blood Subst Immobil Biotechnol 25:163-180.
11. D'Agnillo, F. and T. M. S. Chang. 1998. Absence of hemoprotein-associated free radical events following oxidant challenge of crosslinked hemoglobin-superoxide dismutase catalase. Free Rad Biol Med. 24:906-912.
12. Doherty, D. H., M. P. Doyle, S. R. Curry, R. J. Vali, T. J. Fattor, J. S. Olson, D. D. Lemon. 1998. Rate of reaction with nitric oxide determines the hypertensive effect of cell-free hemoglobin. Nat. Biotechnology 16:672-676.
13. Enright, H. 1997. Cyplex platelet alternative (infusible platelet membranes)-progress and plans. Current advances in platelet substitutes, Preceding the 39^{th} Annual Meeting of the American Society of Hematology, San Diego, Calif., Dec. 5, 1997.
14. Escolar, G., R. Mazzara, R. Castillo, A. Ordinas. 1994. The role of the Baumgartner technique in transfusion medicine: research and clinical applications. Transfusion 34:542-549.
15. Fischer, S. R., D. L. Traber. 1999. L-arginine and endothelin receptor antagonist bosentan counteract hemodynamic effects of modified hemoglobin. Shock. 11:283-90.
16. Galan, A. M., J. Bozzo, M. R. Hernandez, M. Pino, J. C. Reverter, R. Mazzara, G. Escolar, A. Ordinas. 2000. Infusible platelet membranes improve hemostasis in thrombocytopenic blood: experimental studies under flow conditions. Transfusion 40:1074-1080.
17. Gawryl, et al., U.S. Pat. No. 5,691,452 issued Nov. 25, 1997.
18. Gawryl, et al., U.S. Pat. No. 5,808,011 issued Sep. 15, 1998.
19. Hedner, U. 2001. rfVIIa as a hemostatic agent. Semin Hematol. 38:43-47.
20. Jurlander, B. L. Thim, N. K. Klausen, E. Persson, M. Kjalke, P. Rexen, T. B. Jorgensen, P. B. Ostergaard, E. Erhardsten, S. E. Bjorn. 2001. Recobminant activated facter VII (rFVIIa): characterization, manufacturing and clinical development. Semin Thromb Hemost. 27:373-384.
21. Kameneva, M. V., Z. J. Wu, A. Uraysh, B. Repko, K. N. Litwak, T. R. Billiar, M. P. Fink, R. L. Simmons, B. P. Griffith, H. S. Borovetz. 2004. Blood soluble drag-reducing polymers prevent lethality from hemorrhagic shock in acute animal experiments. Biorheology. 41:53-64.
22. Katz, L. M., J. E. Manning, S. McCurdy, L. B. Pearce, M. S. Gawryl, Y. Wang, C. Brown. 2002. HBOC-201 improves survival in a swine model of hemorrhagic shock and liver injury. Resuscitation 54:77-87.
23. Kenet, G., R., Walden, A. Eldad, U. Martinowitz. 1999. Treatment of traumatic bleeding with recombinant factor VIIa. Lancet. 354:1879.
24. Koustova, E., P. Rhee, T. Hancock, H. Chen, R. Inocenciao, A. Jaskville, W. Hanes, C. R. Valeri, H. B. Alam. 2003. Ketone and pyruvate Ringer's solutions decrease pulmonary apoptosis in a rat model of severe hemorrhagic shock and resuscitation. Surger. 134:267-74.
25. Kumar. 1998. Improved blood substitute: evaluation of its effects on human endothelial cells. ASAIO J. 44:M356-367.
26. Liao, J. C., T. W. Hein, M. W. Vaughn, K. T. Huang, L. Kuo. 1999. Intravascular flow decreases erythrocyte consumption of nitric oxide. Proc. Natl Acad Sci (USA) 96:8757-8761.
27. Light, et al., U.S. Pat. No. 5,895,810 issued Apr. 20, 1999.
28. Martinowitz, U., G. Kenet, E. Segal, J. Luboshitz, A. Lubetsky, J. Ingerslev, M. Lynn. 2001 (I). Recombinant activated factor VII for adjunctive hemorrhage control in trauma. J. Trauma 51:431-438.
29. Martinowitz, U., J. B. Holcomb, A. E. Pusateri, M. Stein, N. Onaca, M. Freidman, J. M. Macaitis, D. Castel, U. Hedner, J. R. Hess. 2001 (II). Intravenous rFVIIa administered for hemorrhage control in hypothermic coagulopathic swine with grade V liver injuries. J. Trauma 50:721-729.
30. Rausch, et al. (I). U.S. Pat. No. 5,905,141 issued May 18, 1999.
31. Rausch, et al., (II). U.S. Pat. No. 5,618,919 issued Apr. 8, 1997.
32. Rausch, et al., (III). U.S. Pat. No. 5,084,558 issued Jan. 28, 1992.
33. Rausch, et al., (IV). U.S. Pat. No. 5,296,465 issued Mar. 22, 1994.
34. Rausch, et al., (V). U.S. Pat. No. 5,840,852 issued Nov. 24, 1998.
35. Rausch, et al., (VI). U.S. Pat. No. 5,753,616 issued May 19, 1998.
36. Scigliano, E. 1997. Infusible platelet membranes (Cyplex) for control of bleeding in patients with thrombocytopenia: results of pilot trials. Current advances in platelet substitutes, Preceding the 39^{th} Annual Meeting of the American Society of Hematology, San Diego, Calif., Dec. 5, 1997.
37. Simoni, J., G. Simoni, R. Martinez-Zaguilan, D. E. Wesson, C. D. Lox, S. D. Prien, R. V. Kumar. 1998. Improved blood substitute: evaluation of its effects on human endothelial cells. ASAIO J. 44:M356-367.
38. Sloan, E. P., M. Koenigsberg, D. Gens, M. Cipolle, J. Runge, M. N. Mallory, G. Rodman, Jr. 1999. Diaspirin cross-linked hemoglobin (DCLHb) in the treatment of severe traumatic hemorrhagic shock: a randomized controlled efficacy trial. JAMA 282:1857-1864.
39. Su, D., R. I. Roth, M. Yoshida, J. Levin. 1997. Hemoglobin increases mortality from bacterial endotoxin. Infect Immun. 65:1258-1266.
40. Trunkey, D. D. and M. Slater. 2000. Management of battle casualties. Trauma, 4^{th} ed. K. L. Matteox, eds pg 1211-1228.
41. Wertz, et al., U.S. Pat. No. 5,691,453 issued Nov. 25, 1997.

What is claimed is:

1. A multifunctional blood substitute comprising polymerized hemoglobin and procoagulation factors.

2. The multifunctional blood substitute of claim 1 also comprising one or more pharmacological interventions.

3. The multifunctional blood substitute of claim 1, wherein said polymerized hemoglobin is composed of less that one percent tetrameric hemoblobin.

4. The multifunctional blood substitute of claim 1, wherein said procoagulation factor is factor VIIa.

5. The multifunctional blood substitute of claim 2, wherein said pharmacological interventions are selected from the group consisting of immunomodulators and free radicals.

6. The multifunctional blood substitute of claim 4, wherein said factor VIIa is recombinant factor VIa.

7. The multifunctional blood substitute of claim 4, wherein said procoagulation factors also includes infusible platelet membranes.

8. The multifunction blood substitute of claim 5, wherein said immunomodulators or free radical scavengers are selected from the group consisting of anti-CD 18, pentoxifylline, 2-mercaptopropionyl glycine, mercaptoethylguanidine, edaravone and melatonin.

9. A method of treating patients or providing a whole blood-like bridging volume replacement, comprising replacing blood volume by administering a hemoglobin solution to the patient wherein said hemoglobin solution comprises the multifunctional blood substitute in claim 1.

10. The method of claim 9, wherein said hemoglobin solution also comprises one or more pharmacological interventions.

11. The method of claim 9, wherein said procoagulation factor of said multifunctional blood substitute is factor VIa.

12. The method of claim 9, wherein said polymerized hemoglobin is composed of less that one percent tetrameric hemoglobin.

13. The method of claim 9, wherein said polymerized hemoglobin is gluteraldehyde cross-linked bovine hemoglobin superoxide dismutase catalase.

14. The method of claim 10, wherein said pharmacological interventions are selected from the group consisting of immunomodulators and free radical scavengers.

15. The method of claim 11, wherein said factor VIa is recombinant VIIa.

16. The method of claim 11, wherein said factor VIIa is administered at a dose of 45 ug/kg to 360 μg/kg.

17. The method of claim 11, wherein said procoagulation factors also includes infusible platelet membranes.

18. The method of claim 17, wherein said factor VIIa is administered at a dose of 45 ug/kg to 360 μg/kg and said infusible platelet membranes are administered at 4 mg/kg to 8 mg/kg.

18. The method of claim 10, wherein said immunomodulators and free radical scavengers are selected from the group consisting of anti-CD18, pentoxifylline, 2-mercaptopropionyl glycine, mercaptoethylguanidine, edaravone and melatonin.
